(19) United States
(12) Patent Application Publication
(10) Pub. No.: US 2007/0036858 A1
(43) Pub. Date: Feb. 15, 2007

(54) Drug delivery system for topical administration

(76) Inventor: Maya Schneider, Hofit (IL)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/501,839

(22) Filed: Aug. 10, 2006

Related U.S. Application Data
(60) Provisional application No. 60/706,757, filed on Aug. 10, 2005.

(51) Int. Cl. A61K 9/24

(52) U.S. Cl. 424/471

(57) ABSTRACT

This invention is directed to new drug delivery systems (SMS packaging™) and uses thereof, of freshly prepared, effective, high concentrated, hygienic pharmaceutical compositions. The new delivery systems, including sucralfate, is used for treating and preventing oral diseases in mammals. Moreover this invention provides pharmaceutical compositions, methods and kits for topical administration to oral ulcers comprising sucralfate (Aftapet™ Aftakid™ Aftakit™ Chemokit™). This invention is a platform delivery system for any drug, which needs a fresh mixture of powder and water,

### CROSS REFERENCE TO RELATED APPLICATIONS

This application claims the benefit of U.S. Ser. No. 60/706,757, filed on Aug. 10, 2005 which is incorporated in their entirety herein by reference.

### FIELD OF THE INVENTION

This invention is directed to new delivery systems (SMS packaging™) and uses thereof, of freshly prepared, high concentrated, effective, hygienic pharmaceutical compositions. The new delivery systems, including sucralfate, is used for treating and preventing oral diseases in mammals. Moreover this invention provides pharmaceutical compositions, methods and kits for topical administration to oral ulcers comprising sucralfate (Aftapet™ Aftakit™ Chemokit™)

### BACKGROUND OF THE INVENTION

Recurrent Aphthous Stomatitis (R.A.S) is a very common disease. 20% of the general population suffers from the symptoms of the chronic disease, and 60% of the general population are affected from Recurrent Aphthous Stomatitis during their lifetime. Recurrent Aphthous Stomatitis (R.A.S) is a chronic disease, that may begin at young or older ages, characterized by painful, recurrent superficial oral ulcers. The ulcers appear, enlarge and disappear, usually in 10-14 days on the oral mucosa, and disrupt the oral function such as talking and eating and cause intense pain. Recurrent Aphthous Stomatitis appear in three clinical types: the most common type of R.A.S (80%) is called Minor Aphthae, characterized by one to four ulcers simultaneously in the oral mucosa, 1-5 mm diameter each, which heal spontaneously after 10-14 days, until the next seizure. Another type (8%) is Major Aphthae, characterized by a single ulcer, larger than 1 cm diameter, that causes much pain. Such ulcers, heal only after 6 weeks followed by another Major Aphthae, a condition called “an ulcer follows an ulcer”. A third type is the Herpetiform Lesions (8%) with hundreds of pinpoints painful ulcers that may coalesce to one enormous painful ulcer.

The etiology of the disease is not known. The occurrence of the lesions has been related to family history, emotional stress, dietary deficiencies, seasonal variations, febrile infections, mucosal trauma and food allergy.

Treatment for R.A.S ulcers has not proved beneficial. Local application of Corticosteroids relieves partially the symptoms but are not suitable for a long term treatment, especially not for children because of adverse reactions. Application of local anaesthetic cream or of a local cover of the ulcer provides some protection against trauma or chemical insult. Such pastes are difficult to put on the ulcer and only give partial relief which does not last long as they are washed out by the saliva and by food.

Chemotherapy induced Oral Mucositis is an acute or chronic complication of 1-4 severity degrees of oral and esophageal ulcers, which occur in approximately 40% of patients receiving chemotherapy. Higher rates of 75% occur in patients preparing for BMT (bone marrow transplantation). The incidence is higher in patients getting continuous infusion chemotherapy. 400,000 patients per year develop Oral Mucositis in the USA. Similar figures are shown in the world. Oral Mucositis starts 7-10 days after high dose cancer therapy. Oral Mucositis is self limited when uncomplicated by infection, typically heals within 2-4 weeks after cessation of cytotoxic chemotherapy.

Sucralfate is an aluminum octa-sulfate sucrose salt. Sucralfate was developed as an adjunctive treatment for stomach ulcers in humans. It is minimally absorbed into the body, inhibits the action of pepsin in the presence of stomach acid, and its actions are entirely on the lining of the stomach and duodenum mucosa. Although its mechanism is not entirely understood, it is known that it exerts its effect through a local, rather than systemic, action.

There is a need in the art to develop an efficient treatment for Recurrent Aphthous Stomatitis.

### SUMMARY OF THE INVENTION

In one embodiment, this invention provides a drug delivery system, comprising a first unit of a liquid and a second unit of a powdered drug, wherein said first unit is positioned inside said second unit.

In one embodiment, this invention provides a use of a drug delivery system for topical administration of a pharmaceutical composition, comprising a first unit of a liquid , and a second unit of a drug, and a tube attached to said second unit; wherein said first unit is positioned inside said second unit, comprising the following steps:
a. allow said liquid to mix with said drug in said second unit, thereby preparing a pharmaceutical composition comprising a solution or a paste of said drug;
b. convey said pharmaceutical composition through said tube; and
c. administer topically said pharmaceutical composition.

In one embodiment, this invention provides a use of a drug delivery system, which is formulated to be safe for ingestion, for topical administration of a pharmaceutical composition, comprises a first unit of a liquid, and a second unit of a drug; wherein said first unit is positioned inside said second unit, comprising the following steps:
a. allow said liquid to mix with said drug in said second unit, thereby preparing a pharmaceutical composition comprising a solution or a paste of said drug;
b. administer topically said pharmaceutical composition.

In another embodiment, this invention provides the use of the drug delivery systems of this invention for treating an ulcer of Aphthous Stomatitis, in a subject, comprising the steps of mouth rinse followed by topically administration of the pharmaceutical composition, wherein the drug is sucralfate, and the liquid is an aqueous solution.

In another embodiment, this invention provides the use of the drug delivery systems of this invention for treating an ulcer of Aphthous Stomatitis in a subject suffering from Oral Mucositis, induced by chemotherapy or radiotherapy, comprising the steps of mouth rinse followed by topically administration of the pharmaceutical composition, wherein said drug is sucralfate, and said liquid is an aqueous solution.

In another embodiment, this invention provides the use of the drug delivery systems of this invention for treating an ulcer of Oral Lichen Planus in a subject, comprising the steps of mouth rinse followed by topically administration of the pharmaceutical composition,wherein the drug is sucralfate, and the liquid is an aqueous solution.

In one embodiment, this invention provides a topical composition for topical treatment for ulcers of Aphthous Stomatitis comprising sucralfate in an aqueous solution or paste, wherein the concentration of the sucralfate is in a range of ½-10 gr in about 2-5 mL water and isotonic saline, freshly prepared and freshly administered.

In one embodiment, this invention provides a kit for treating ulcers of Aphthous Stomatitis comprising sucralfate powder.

In one embodiment, this invention provides a method of treating ulcer of Aphthous Stomatitis, periodontal pockets, abscesses, site of extractions or any combination thereof in a subject, comprising the steps of:
a. mouth-rinse
b. mixing a powder comprising sucralfate with water and isotonic saline to obtain a mixture; and
c. topically freshly administering the mixture to said ulcer and surrounding mucosa;
   thereby treating an ulcer of Aphthous Stomatitis in said subject.

In one embodiment, this invention provides a method of preventing the recurrence of an ulcer of Aphthous Stomatitis in a subject suffering from an ulcer of Aphthous Stomatitis, comprising the steps of;
a. mouth rinse
b. mixing a powder comprising sucralfate with water and isotonic saline to obtain a mixture; and
c. topically freshly administering the mixture to said ulcer and surrounding mucosa;
   thereby preventing the recurrence of an ulcer of Aphthous Stomatitis in said subject.

In one embodiment, this invention provides a method of treating an ulcer of Aphthous Stomatitis in a subject suffering from Oral Mucositis, induced by chemotherapy or radiotherapy, comprising the steps of;
a. mouth rinse
b. mixing a powder which includes sucralfate with water and isotonic saline to obtain a mixture; and
c. topically freshly administering the mixture to the ulcer and surrounding mucosa;
   thereby treating an ulcer of Oral Mucositis, induced by chemotherapy or radiotherapy.

In one embodiment, this invention provides a method of treating an ulcer of Oral Lichen Planus in a subject, comprising the steps of;
a. applying a rinsing fluid on the ulcer;
b. mixing a powder which includes sucralfate with water and isotonic saline to obtain a mixture; and
c. topically freshly administering the mixture to the ulcer.
   thereby treating an ulcer of Oral Lichen Planus in a subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic depiction of the drug delivery system. The first unit (1-10) of the liquid is a bowl shaped base, wherein a wall (1-50) separates between the two units and a ring connecting (1-60) the base and the separating wall. Thus, providing enough toughness for stability of the wall but also enough weakness so it can be pressed by hand. This system is sealed by a cover made of LDPE (Low Density Polyethylene) Pressing the first unit (inside bubble)- one may smash, a weak barrier which allows the liquid to burst out from the first unit into the second unit (1-20). The second unit has the entire shape of the package. The liquid mixes with the powder by 3-4 manual pressing. At the end of the second unit there's a small funnel (1-40). Through that funnel the pharmaceutical composition is topically administered. The funnel is closed by a removable cup (1-30), wherein, the cup is removed, and “glued” to the outer wall, not interfering to administering the pharmaceutical composition nor getting lost. In one embodiment, 3 mL of liquid is added to the first unit, and is between about 1-3 gr of powder is added to the second unit.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Sucralfate, a salt of alpha-D-glucopyranoside, beta-D fructosfuranosyl-octakis-(hydrogen sulfate) aluminum complex, is presented hereby as an efficient medicine, when it is prepared as a concentrated mixture. The increased concentration may form higher linkage between the salt and the exposed proteins of the ulcer and provide a more potent drug, for treatment of ulcers in the oral mucosa (R.A.S and chemo-induced Oral Mucositis).

This invention provides, in some embodiments, kits, methods, compositions, and drug delivery systems for treating ulcer of Aphthous Stomatitis, periodontal pockets, abscesses, site of extractions or any combination thereof in a subject. In some embodiment, such kits, methods, compositions, and drug delivery systems are useful in preventing the recurrence of an ulcer of Aphthous Stomatitis. In some embodiment, such kits, methods, compositions, and drug delivery systems are useful in treating an ulcer of Aphthous Stomatitis in a subject suffering from Oral Mucositis, induced by chemotherapy or radiotherapy. In another embodiment such kits, methods, compositions, and drug delivery systems are useful in treating an ulcer of Oral Lichen Planus.

According to this aspect, and in one embodiment, such kits, methods, compositions, and drug delivery systems are useful in treating mammals, humans, including children and pets.

In some embodiments, the methods, kits, compositions and drug delivery systems of this invention may be used for preparing a freshly prepared pharmaceutical composition. In one embodiment, the drug delivery systems may be used for storing drugs for long periods. In another embodiment the long periods are between one month to 3 years. In another embodiment, said long periods are between one month to five years. In another embodiment, the drug delivery system, comprises a composition that does not require preservatives or cooling in order to provide storage stability.

In another embodiment, the methods, kits, compositions and drug delivery systems of this invention may be used for preparing a concentrated pharmaceutical composition. In another embodiment, the drug delivery systems of this invention may be used for preparing a paste. In another embodiment, the drug delivery system is a disposable system. In another embodiment the use of the drug delivery systems allow hygienic treatment. In another embodiment, kits, methods and drug delivery systems comprise a composition which may include mainly active ingredients, without preservatives. In another embodiment, there is not known adverse reactions.

In another embodiment the concentrated mixture paste comprising, for example, sucralfate, is a more potent drug for treating an oral disease, for soothing effect on the mucosa, for preventing new ulcers and for providing immediate relief of pain.

In another embodiment, the drug delivery systems of this invention may be used for preparing hygienic composition, and hygienic administration of a pharmaceutical composition.

In one embodiment, the compositions, methods, kits, drug delivery systems or the use in the methods of this invention are topically administered. In another embodiment, the compositions, methods, kits, drug delivery systems or the use in the methods of this invention are administered on an oral mucosa. In another embodiment, the compositions, methods, kits, drug delivery systems or the use in the methods of this invention are being used such as, for example, as a chewing gum, and thus administering a pharmaceutical composition on an oral mucosa. In another embodiment, the compositions, methods, drug delivery systems or the use in the methods of this invention are further ingested. In another embodiment, the compositions, and drug delivery systems are made of a digestible materials.

In one embodiment, the drug delivery systems or the use in the methods of this invention comprise a first unit of a liquid and a second unit of a drug, wherein said first unit is positioned inside said second unit. In another embodiment, the drug delivery system of this invention is a Smash Mix Squeeze (SMS) packaging. In another embodiment, the two units are bound, fixed, attached or connected to each other (similar to a yolk in the egg).

In one embodiment, the drug delivery systems of this invention are made from silicon, polyesther, aluminum, plastic, high density polyethylene (HDPE), low density polyethylene (LDPE), or digestible material like candy, gelatin, soft-gel, guar-gum, bubble-gum, -poly-β-hydroxybutyrate (PHB) or any combination thereof.

In one embodiment, the drug delivery systems of this invention, further comprise a tube, or a funnel attached to the second unit. In another embodiment, the pharmaceutical composition is applied via the tube or funnel, and topically administered. In another embodiment, the pharmaceutical composition is conveyed from the second unit to the funnel or tube by applying pressure on the second unit. In another embodiment, the pharmaceutical composition is conveyed from the second unit to the funnel or tube by squeezing the second unit.

In one embodiment, the compositions, methods, kits, drug delivery systems or the use in the methods of this invention make use of a liquid, wherein the liquid is water. In another embodiment, the liquid is an aqueous solution. In another embodiment, the liquid is an aqueous solution, comprising isotonic saline solution and water in a range of between about 1:1 to 8:1. In another embodiment, the liquid is an aqueous solution, comprising isotonic saline solution and water in a 1:1 ratio. In another embodiment, the liquid is an aqueous solution, comprising isotonic saline solution and water in a 2:1 ratio respectively. In another embodiment, the liquid is an aqueous solution, comprising isotonic saline solution and water in a 1:2 ratio respectively. In another embodiment, the liquid is an aqueous solution, comprising isotonic saline solution and water in a 1:3 ratio respectively. In another embodiment, the liquid is an aqueous solution, comprising isotonic saline solution and water in a 1:4 ratio respectively. In another embodiment, the liquid is an aqueous solution, comprising isotonic saline solution and water in a 3:1 ratio respectively. In another embodiment, the liquid is an aqueous solution, comprising isotonic saline solution and water in a 4:1 ratio respectively.

In one embodiment, the term “isotonic” saline solution refers to 0.9% w/v sodium chloride aqueous solution.

In another embodiment, the liquid is an aqueous solution comprising a second drug. In another embodiment the second drug is antibiotics. In another embodiment, the aqueous solution comprises an anesthetic material. In another embodiment, the liquid is an aqueous solution comprising an aromatic oil. In another embodiment aromatic oil is sweet marjoram, myrrh, vanilla, lavender, juniper, clove bud or any combinations thereof. In another embodiment, the liquid further comprises a flavor material. In another embodiment, the aqueous solution comprises any compound for homogeneity and creamy texture such as, for example, aromatic oils.

In one embodiment, the compositions, methods, kits, drug delivery systems or the use in the methods of this invention make use of a drug. In another embodiment, the drug is a powder. In another embodiment, the drug is sucralfate. In another embodiment, the drug is an antibiotic powder. In another embodiment, the drug is a combination of two drugs. In another embodiment, the drug is a mixture of sucralfate and antibiotics. In another embodiment, the drug is a combination of sucralfate and an anesthetic material.

In one embodiment, the compositions, methods, kits, drug delivery systems or the use in the methods of this invention make use of an aqueous solution, comprising water and isotonic saline solution in a 1:1 ratio, and of sucralfate.

In one embodiment, the compositions, methods, kits, of this invention make use of a drug delivery system, for topical administration of a pharmaceutical composition, comprising a first unit of a liquid, and a second unit of a drug, and a tube attached to the second unit; wherein the first unit is positioned inside the second unit, comprising the following steps:
a. allow the liquid to mix with the drug in the second unit, thereby preparing a pharmaceutical composition comprising a solution or a paste of the drug;
b. convey the pharmaceutical composition through the tube; and
c. administer topically the pharmaceutical composition.

In another embodiment, step (a) is performed not more than one hour prior to step (c). In another embodiment, the short timing between the preparation and the administration of pharmaceutical composition allow preparation of a fresh and reactive composition.

In one embodiment, the compositions, methods, kits, make use of the drug delivery systems. In another embodiment, pressure is applied on the first unit comprising liquid, to allow the liquid to pass into the second unit and mix with the drug. In another embodiment the pressure is applied by the hands, to allow the liquid to pass into the second unit and mix with the drug. In another embodiment, the first unit is smashed by the hands to allow the liquid to pass into the second unit and mix with the drug.

In one embodiment, the second unit is squeezed by hands, conveying the pharmaceutical composition through the tube or funnel and manually administered on the oral mucosa, for example using a spatula.

According to this aspect, and in some embodiments, the drug delivery system, wherein the composition is administered manually, is made from silicon, polyesther, aluminum or polyethylene.

In one embodiment, the compositions, methods, kits, of this invention make use of a drug delivery system formulated to be safe for ingestion, for topical administration of a pharmaceutical composition, comprising a first unit of a liquid, and a second unit of a drug; wherein the first unit is positioned inside said second unit, comprising the following steps:
a. allow the liquid to mix with the drug in the second unit, thereby preparing a pharmaceutical composition comprising a solution or a paste of the drug;
b. spread topically said pharmaceutical composition.

In another embodiment, step (a) is performed not more than one hour prior to step (b). In another embodiment, the short timing between the preparation and the administration of pharmaceutical composition allow preparation of a fresh and reactive composition.

In another embodiment, the drug delivery systems are being applied by chewing the drug delivery system and allowing the liquid and the drug to mix in the mouth cavity and topically spread the composition on the ulcer site. In another embodiment, one may chew the drug delivery system between 4 to 5 times and further eject it. In another embodiment, one may chew the drug delivery system between 4 to 5 times and further ingest it. In another embodiment, one may chew the drug delivery system between 1 minute to 2 minutes and then eject or ingest it.

In one embodiment, the drug delivery system is made from a digestible material such as, for example a candy, a soft gel, a chewing gum or any combination thereof, for treating ulcers in the mouth cavity. According to this aspect, and in some embodiments, the use of the drug delivery system comprises chewing the drug delivery system and allowing the liquid to mix with the drug and spread on the ulcer site in the mouth cavity. In one embodiment, the digestible material may be ingested, melted or ejected. According to this aspect, and in some embodiments, the drug delivery is made from gelatin, gel-capsule, guar-gum, candy, poly-β-hydroxybutyrate, optionally with homogenizer like aromatic oil, or any combination thereof. In another embodiment, the liquid in the drug delivery system may further comprise a flavor material.

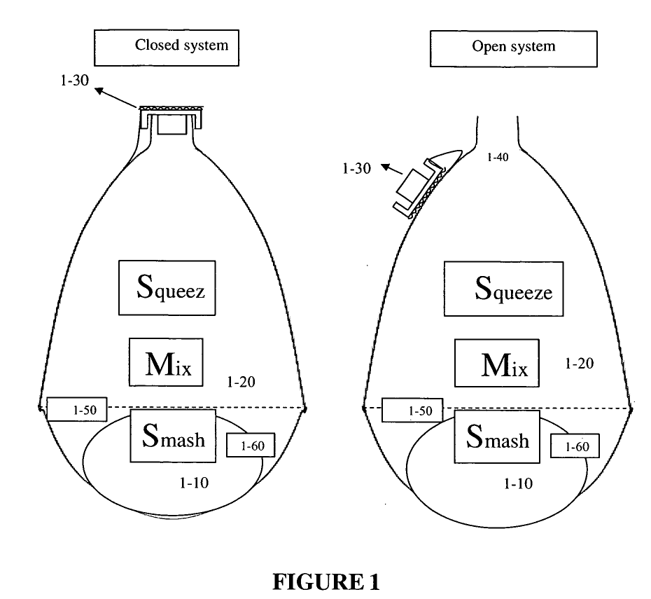

One embodiment of an envisioned drug delivery system is depicted in FIG. 1. The first unit (1-10) of the liquid is a bowl shaped base, wherein a wall (1-50) separates between the two units and a ring connecting (1-60) the base and the separating wall. Thus, providing enough toughness for stability of the wall but also enough weakness so it can be pressed by hand. This system is sealed by a cover made of LDPE (Low Density Polyethylene). Pressing the first unit (inside bubble)- one may smash a weak barrier which allows the liquid to burst out from the first unit . into the second unit (1-20). The second unit has the entire shape of the package. The liquid mixes with the powder by 3-4 manual pressing. At the end of the second unit there's a small funnel (1-40). Through that funnel the pharmaceutical composition is topically administered. The funnel is closed by a removable cup (1-30), wherein, the cup is removed, and “glued” to the outer wall, not interfering to administering the pharmaceutical composition nor getting lost. In one embodiment, 3 mL of liquid is added to the first unit, and is between about 1-3 gr of powder is added to the second unit.

It is to be understood that, any embodiment described herein, for example, with respect to the, first unit, second unit, liquid, drug, and sucralfate will be applicable to any aspect of this invention, including methods, kits, drug delivery system or composition and represent embodiments thereof.

In one embodiment, the compositions, methods, kits, drug delivery systems of this invention are useful in treating an ulcer of Aphthous Stomatitis, in a subject, comprising the steps of mouth rinse followed by topically administration of the pharmaceutical composition, wherein the drug is sucralfate, and the liquid is an aqueous solution with isotonic saline. In another embodiment, Aphthous Stomatitis is Recurrent Aphthous Stomatitis (R.A.S), or Oral Mucositis , chemo-induced, oral ulcers of Oral Lichen Planus, or any combination thereof. In another embodiment, the Aphthous Stomatitis occur as a minor Aphthae, major Aphthae or Herpetiform Lesions.

In one embodiment, the compositions, methods, kits, drug delivery systems of this invention are useful in treating an ulcer of Aphthous Stomatitis in a subject suffering from Oral Mucositis, induced by chemotherapy or radiotherapy, comprising the steps of mouth rinse followed by topically administration of the pharmaceutical composition, wherein the drug is sucralfate, and the liquid is an aqueous solution. In another embodiment, the Oral Mucositis is induced by chemotherapy, by radiotherapy or induced by preparing the body for bone-marrow transplantation.

In one embodiment, the compositions, methods, kits, drug delivery systems of this invention are useful in preventing the recurrence of an ulcer of Aphthous Stomatitis in a subject comprising the steps of applying a rinsing fluid on the ulcer followed by topically administration said pharmaceutical composition, wherein the drug is sucralfate, and the liquid is an aqueous solution.

In one embodiment, the compositions, methods, kits, drug delivery systems of this invention are useful in treating an ulcer of Oral Lichen Planus Stomatitis in a subject comprising the steps of applying a rinsing fluid on the ulcer followed by topically administration said pharmaceutical composition, wherein the drug is sucralfate, and the liquid is an aqueous solution with isotonic saline.

In one embodiment, the compositions, methods, kits, drug delivery systems of this invention are useful in stopping the development of an ulcer of Aphthous Stomatitis in a subject comprising the steps of applying a rinsing fluid on the ulcer followed by topically administration said pharmaceutical composition, wherein the drug is sucralfate, and the liquid is an aqueous solution with isotonic saline.

In one embodiment of the invention, the term “Aphthous Stomatitis” refers in one embodiment to Recurrent Aphthous Stomatitis (RAS), in another embodiment to Major Aphthous Stomatitis (MAS), in another embodiment to Recurrent Aphthous Ulcers (RAU) and, in another embodiment to Major Aphthous Ulcers (MAU), depending on the frequency or severity of the outbreaks. Particularly vulnerable are the areas that rub against the teeth and any spot subject to trauma. In one embodiment, the term “Aphthous Stomatitis” refers to Oral Mucositis, induced by chemotherapy treatment for cancer patients. In another embodiment Oral Mucositis induced by radiotherapy for example for head or neck cancer. In another embodiment, Oral Mucositis induced by preparing treatment for Bone Marrow Transplantation (BMT).

In one embodiment, the drug delivery systems, kits, and methods make use of a rinsing fluid prior to administering the pharmaceutical composition. In another embodiment, the rinsing fluid contains Aromatic Oils which disinfect the ulcer before applying the pharmaceutical composition on it, thus providing a better healing effect on the ulcer. In another embodiment the rinsing fluid comprises of a water and aromatic oil mixtures. In another embodiment the rinsing fluid comprises of pure water or in another embodiment water with an etheric oil.

In one embodiment, the compositions, methods, kits, drug delivery systems of this invention make use of an aqueous solution and of sucralfate. In another embodiment, the concentration of the sucralfate may be in a range of ½-10 gr in about 2-5 mL water and isotonic saline in a range of 1:1 to 1:8 ratio. In another embodiment, the concentration of the sucralfate may be in a range of ½-10 gr in about 2-5 mL water and isotonic saline in 1:1 ratio. In another embodiment the concentration of sucralfate may be in the range of 1-5 gr sucralfate in 1-5 mL water and isotonic saline in a range of 1:1 to 2:1 ratio. In another embodiment the concentration of sucralfate may be in the range of 5-10 gr sucralfate in 1-5 mL water and isotonic saline in 1:1 to 2:1 ratio. In another embodiment, the concentration of sucralfate may be in the range of 1-2 gr sucralfate in 1-2 mL water and isotonic saline in 1:1 to 2:1 ratio. In another embodiment, the concentration of sucralfate may be in the range of 2-2.5 gr sucralfate in 1 mL water and isotonic saline in 1:1 to 2:1 ratio. In another embodiment, the concentration of sucralfate may be in the range of 2.5-3 gr sucralfate in 1-2 mL water and isotonic saline in a 1:1 to 2:1 ratio. In another embodiment the concentration of sucralfate may be in the range of 3-4 gr sucralfate in 1-3 mL water and isotonic saline in a 1:1 to 1:8 ratio. In another embodiment the concentration of sucralfate may be in the range of 4-5 gr sucralfate in 1 mL water and isotonic saline in 1:1 to 2:1 ratio. In another embodiment the concentration of sucralfate is 2 gr in a range of between about 1-2 mL water and isotonic saline in 1:1 ratio. In another embodiment the concentration of sucralfate is 2 gr in a range of between about 1-2 mL water and isotonic saline in 2:1 ratio. In another embodiment the concentration of sucralfate is 2 gr in a range of between about 1-2 mL water and isotonic saline in 1:2 ratio. In another embodiment, the aqueous solution is about ½ mL isotonic saline solution and about 2 mL of water. In another embodiment, the composition further comprises an acceptable carrier and/or excipient.

In one embodiment, this invention provides a topical composition for topical treatment of ulcers of Aphthous Stomatitis comprising sucralfate in an aqueous solution or paste or cream, wherein the concentration of the sucralfate is in a range of ½-10 gr in about 2-5 mL water and isotonic saline in a 1:1 ratio, freshly prepared and freshly administered.

In another embodiment, the term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers.

It is to be understood that the compositions of this invention and any embodiments described herein, with respect to the compositions will be applicable to any aspect of this invention, including methods, kits, drug delivery sysytem and represent embodiments thereof.

In one embodiment, the composition of this invention is a paste, wherein the concentration of the sucralfate in the liquid is as such that forms a paste (Aftakit). In another embodiment the composition is a gel. In another embodiment, the composition is a solution, wherein homogenizer optionally may be added (Chemokit). In another embodiment the composition is in a texture of cream.

In one embodiment the methods, kits, drug delivery system and compositions are used for treating or preventing an ulcer of a subject. In another embodiment, the term “subject” refers to a mammal, human including children or a pet.

In one embodiment, this invention provides a method of preventing the recurrence of an ulcer of Aphthous Stomatitis in a subject, comprising the step of topically administering to the ulcers of Aphthous Stomatitis of the subject a pharmaceutical composition, wherein the composition is in a form of a paste, cream, gel, ointment or lotion.

In one embodiment the methods kits, and drug delivery system of this invention make use of a composition, wherein the composition is in a form of a paste, cream, gel, ointment or lotion. In another embodiment, the composition comprises aromatic oil.

In another embodiment, the aromatic oil is Sweet Marjoram, Myrrh, Vanilla, Lavender, Juniper, Clove bud or other or any combinations thereof. In another embodiment the composition may further comprise a drop of Sweet Marjoram, a drop of myrrh, a drop of vanilla, a drop of lavender, a drop of juniper, a drop of clove-bud or any combinations thereof.

According to this aspect, and in some embodiments, the methods, kits and drug delivery system make use of a pharmaceutical composition in a form of a powder, paste, capsule, tablet, cream, gel, suspension, gum, candy, ointment or lotion. In another embodiment, the pharmaceutical composition is freshly prepared. In another embodiment the pharmaceutical composition does not include preservatives. In another embodiment the pharmaceutical composition includes mainly active ingredients. In another embodiment natural flavors may be added for example banana, strawberry or vanilla tastes. In another embodiment anesthetic agents may be added.

In one embodiment, this invention provides a kit for treating ulcers of Aphthous Stomatitis comprising sucralfate powder.

In one embodiment, the term “kit” refers to a packaged product, which comprises a container of rinsing solution, sucralfate and liquid, stored in individual containers, or a single container, at pre-determined ratios and concentration, for topical adminestration, for which the use of the kit has been optimized, as will be appreciated by one skilled in the art.

According to this aspect, and in one embodiment, the sucralfate powder is kept in a sealed sterile container. In another embodiment the container is opaque and sealed against sun light. In another embodiment, the container is divided into portions wherein each portion contains a predetermined amount of said powder. In another embodiment, the sucralfate should not be exposed to sun light, to prevent activation and decomposition of the drug. In another embodiment, the predetermined amount is ½, 1, 2, or 3 gr for a dose, or 2, 4, 6, or 8 gr for one day treatment, until healing, Bis in Die (BID) or Quater in Die (QID)

In one embodiment, the kit further comprises an aqueous solution wherein said aqueous solution is separated from said powder. In another embodiment, the aqueous solution is divided into portions wherein each portion contains a predetermined amount of said aqueous solution.

In one embodiment the aqueous solution is mineral water. In another embodiment, the aqueous solution is tap water. In another embodiment the aqueous solution is tap water and saline solution.

In one embodiment, the kit of this invention further comprises an applicator. In another embodiment, the kit comprises a rinsing fluid. In another embodiment, the kit comprises aromatic oil.

In one embodiment, the kit will contain instructions for a range of uses of the individual components, which may be present in the kit at various concentrations and/or ratios, in individually marked containers, whereby the end-user is provided optimized instructions for use in a particular application.

In one embodiment, the kits are comprised of agents whose composition and/or concentration are optimized for the types of ulcers for which the kits will be put to use, for example, for treating oral mucosa. In another embodiment, the kits are comprised of agents whose composition and/or concentration are optimized for use in each particular stage of the ulcer.

In one embodiment this invention provides kits for treating Aphthous Stomatitis in humans (Aftakit™), in pets (Aftapet™), or in humans when Aphthous Stomatitis is induced by chemotherapy (Chemokit™).

In one embodiments, the kits, compositions, and methods of this invention are used for treating ulcer of Aphthous Stomatitis, periodontal pockets, abscesses, site of extractions or any combination thereof in a subject, comprising the steps of:
a. mouth-rinse
b. mixing a powder comprising sucralfate with water and isotonic saline to obtain a mixture; and
c. topically administering the fresh mixture to the ulcer and surrounding mucosa;
   thereby treating an ulcer of Aphthous Stomatitis in said subject.

In one embodiments, the kits, compositions, and methods of this invention are used for treating ulcer of Aphthous Stomatitis in a subject suffering from Oral Mucositis, induced by chemotherapy or radiotherapy, comprising the steps of;
a. mouth rinse
b. mixing a powder comprising sucralfate with water and isotonic saline to obtain a mixture; and
c. topically to administer the fresh mixture to the ulcer site and surrounding mucosa;
   thereby treating an ulcer of Oral Mucositis, induced by chemotherapy or radiotherapy.

In one embodiment, the kits, compositions, and methods of this invention are used for preventing the recurrence of an ulcer of Aphthous Stomatitis in a subject suffering from Aphthous Stomatitis, comprising the steps of;
a. mouth rinse
b. mixing a powder comprising sucralfate with water and isotonic saline to obtain a mixture; and
c. topically administering the fresh mixture to the ulcer site in the prodrome and to the surrounding mucosa;
   thereby preventing the recurrence of ulcers of Aphthous Stomatitis.

In one embodiments, the kits, compositions, and methods of this invention are used for treating an ulcer of Oral Lichen Planus in a subject, comprising the steps of;
a. applying a rinsing fluid on said ulcer;
b. mixing a powder comprising sucralfate with water and isotonic saline to obtain a mixture e; and
c. topically administering the fresh mixture to the ulcer site and to the surrounding mucosa;
   thereby treating an ulcer of Oral Lichen Planus in a subject.

In one embodiment, the methods for treating Aphthous Stomatitis, Oral Mucositis, induced by chemotherapy or radiotherapy, Lichen Planus, or preventing Aphthous Stomatitis, make use of a sucralfate and water-Saline mixture. In another embodiment the concentration of the sucralfate is in a range of ½-10 gr in about 2-5 mL water and isotonic saline in 1:1 ratio. In another embodiment, the concentration of said sucralfate is about 2 gr in a range of between about 1-2 mL water and isotonic saline in a range of 1:1 to 1:8 ratio.

In one embodiment, the methods for treating Aphthous Stomatitis, Oral Mucositis induced by chemotherapy or raditherapy, Oral Lichen Planus, or preventing Aphthous Stomatitis comprise of a mixing step and topically administering step. In another embodiment, the mixing step is performed not more than one hour prior to the topically administration step.

This aspect of the invention will now be described in greater details by reference to the following non-limiting Examples.

### EXAMPLES

### Example 1

### A kit for treating Aphthous Stomatitis

The kit:

A kit for treating and preventing any kind of Aphthous Stomatitis includes 20 bubble-containers and an applicator. Each container was opaque and contained 1 gr sucralfate and an inner container with 1 ml mineral water with aromatic oil sweet Marjoram.

Methods:

After good mouth-rinse with tap water, the inner container was pressurized and the water in the inner container and the powder were mixed and formed a fresh paste. The container was opened and the paste was administered directly or by using a spatula on the ulcer. The treatment was repeated four times a day for a period of five days. The patients filled a diary, over half a year or 4 ulcers.

Results:

30 patients tested the “fresh paste” after good oral rinse. After 1 administration there was a great relief of pain. After 1-2 administration, the ulcer was not felt in the mouth, the healing continued. 96% of the patients reported relief and accelerated healing of the ulcer. 20% reported long remission after treatment of 2 administrations. The kit was fun to use. No appearance of adjacent Aphthae, were observed.

### Example 2

### A kit of syringe and a cup for treating Aphthous Stomatitis

The kit:

A kit for treating and preventing any kind of Aphthous Stomatitis includes 20 bottles of rinsing mineral water, 20 syringes of mineral water, 20 cups of powder and a stick for mixing and administering. Each syringe contained water and isotonic saline 1:1, and each cup included 1 gr of sucralfate .

Methods:

0.5 mL of water-saline was injected to each cup, and the water and the powder of sucralfate were mixed with a stick and administered on the Aphthous ulcers, after a mouth rinse. The drug was administered upon appearance of an ulcer.

Results:

20 patients used the “daily dosage in the cup”. The pain relief was after a day. The healing of ulcer started immediately in 90%.

### Example 3

### A kit for treating Aphthous Stomatitis.

The kit:

A kit for treating and preventing any kind of Aphthous Stomatitis includes 20 gr Sucralfate powder in dark/coloured bottles, a measuring spoon for 1 gr powder, 20 sealed cups with 0.5 mL mineral water and a spatula.

Methods:

1 gr of sucralfate was measured with the measuring spoon and added to the cup of 0.5 cc water, the sucralfate and water were mixed by a spatula and administered on the Aphthous ulcer, immediatly after rinsing the mouth with water and aromatic oil Lavender. 15 adults and 5 children used this kit for half a year. They were instructed to use it already when a “prodromal tingling” of the ulcer site appears and fill a diary. The parents were instructed to administer the medication as soon as there is pain.

Results:

The pain relief was felt by all patients after 1 day. The children responded very quickly, after 1-2 administrations. The ulcers healed after 2-3 days. Remission reported longer than usually. 95% reported relief of symptoms. When treated in the prodrome, the ulcer disappeared before it developed.

### Example 4

### A method of treating Aphthous Stomatitis

The kit:

A kit for treating or preventing any kind of Aphthous Stomatitis includes a colourful sealed container comprising sucralfate, and cotton sticks.

Methods:

The mouth was rinsed with tap water. The cotton stick was wetted with tap water and applied into the sucralfate powder and administered on the Aphthous ulcer. The paste has no taste or smell or feeling of stickiness. The excess is being dispersed in the mouth, forming a protective layer on the mucosa, preventing the recurrence of Aphthous ulcer.

Results:

90% had an immediate relief of pain. The ulcer lasted 3 days in average. The texture was not pleasant.

### Example 5

### A method of treating Aphthous Stomatitis induced by chemotherapy

Composition:

1.5 gr of Sucralfate was mixed with 2 mL water and isotonic saline in a 1:1 ratio, forming a paste.

Methods:

5 female patients with painfull oral stomatitis induced by chemotherapy, were treated with Sucralfate paste, administered 4-6 times a day till recovery. The medication was used when the ulcers were already developed.

Results:

All 5 reported great relief from pain and discomfort and shortening of the ulcer duration. They used it as long as needed.

### Example 6

### A method of treating Aphthous Stopmatitis in pets

Composition:

4 gr of sucralfate dissolved in 4 cc of water.

Methods:

The paste was administerd twice a day, by spatula to 2 dogs, in a veterinarian hospital.

Results:

One dog had a developed Aphthous ulcer, the other had big erosive ulcer in the esophagus. The first showed ulcer minimizing in 2 days, the other didn't show rapid healing.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What i s claimed is:

1. A drug delivery system, comprises a first unit of a liquid and a second unit of a drug, wherein said first unit is positioned inside said second unit.

2. The drug delivery system of claim 1, wherein said first unit and second unit are made from silicon, polyesther, polyethylene, aluminium, gelatin, gel-capsule, guar-gum, candy or any combination thereof.

3. The drug delivery system of claim 1, wherein said units are bound to each other.

4. The drug delivery system of claim 1, wherein said liquid is water.

5. The drug delivery system of claim 1, wherein said liquid is an aqueous solution.

6. The drug delivery system of claim 5, wherein said liquid is an aqueous solution, comprises isotonic saline solution and water in a range of between about 1:1 to 8:1 ratio.

7. The drug delivery system of claim 1, wherein said liquid is an aqueous solution comprising a second drug.

8. The drug delivery system of claim 1, wherein said drug is powder.

9. The drug delivery system of claim 1, further comprises a tube from said second unit, for topical administration.

10. The drug delivery system of claim 1, wherein said drug is sucralfate.

11. The drug delivery system of claim 1, wherein said liquid is an aqueous solution, comprising an isotonic saline solution and water in a range of between about 1:1 to 8:1 ratio, and said drug is sucralfate.

12. Use of a drug delivery system, for topical administration of a pharmaceutical composition, comprises a first unit of a liquid, and a second unit of a drug, and a tube from said second unit;
wherein said first unit is positioned inside said second unit, comprising the following steps:
a. allow said liquid to mix with said drug in said second unit, thereby preparing a pharmaceutical composition comprising a solution or a paste of said drug;
b. convey said pharmaceutical composition through said tube; and
c. administer topically said pharmaceutical composition.

13. The use of claim 12, wherein step (a) is performed not more than one hour prior to step (c).

14. The use of claim 12, wherein pressure is applied on said first unit to allow said liquid to mix with said drug.

15. The use of claim 14, wherein said pressure is applied by hands.

16. The use of claim 12, wherein said units are made from silicon, polyesther, polyethylene, aluminium, gelatin, digestible material or any combination thereof.

17. The use of claim 12, wherein said liquid is water.

18. The use of claim 12, wherein said liquid is an aqueous solution.

19. The use of claim 18, wherein said liquid is an aqueous solution comprising isotonic saline solution and water in a range of between about 1:1 to 8:1 ratio.

20. The use of claim 12, wherein said liquid is an aqueous solution comprising a second drug.

21. The use of claim 12, wherein said drug is powder.

22. The use of claim 12, wherein said drug is sucralfate.

23. The use of claim 12, wherein said liquid is an aqueous solution comprising a isotonic saline solution and water in a range of between about 1:1 to 8:1 ratio, and said drug is sucralfate.

24. The use of claim 12, for treating an ulcer of Aphthous Stomatitis, in a subject, comprising the steps of mouth rinse followed by topically administration of said pharmaceutical composition, wherein said drug is sucralfate, and said liquid is an aqueous solution.

25. The use of claim 12, for treating an ulcer of Aphthous Stomatitis in a subject suffering from Oral Mucositis, induced by chemotherapy or radiotherapy, comprising the steps of mouth rinse followed by topically administration of said pharmaceutical composition, wherein said drug is sucralfate, and said liquid is an aqueous solution.

26. The use of claim 12, for treating an ulcer of Oral Lichen Planus in a subject, comprising the steps of applying a rinsing fluid on the ulcer followed by topically administration of said pharmaceutical composition, wherein said drug is sucralfate, and said liquid is an aqueous solution with isotonic saline.

27. The use of claim 24 wherein the concentration of said sucralfate is in a range of ½-10 gr in about 2-5 mL water and isotonic saline solution in 1:1 ratio.

28. The use of claim 24, wherein said Aphthous Stomatitis is Recurrent Aphthous Stomatitis (R.A.S), or Oral Mucositis or combination thereof.

29. The use of claim 24, wherein said Aphthous Stomatitis occur as a minor Aphthae, major Aphthae or Herpetiform Lesions.

30. Use of a drug delivery system, formulated to be safe for ingestion, for topical administration of a pharmaceutical composition, comprises a first unit of a liquid, and a second unit of a drug;
wherein said first unit is positioned inside said second unit, comprising the following steps:
a. allow said liquid to mix with said drug in said second unit, thereby preparing a pharmaceutical composition comprising a solution or a paste of said drug;
b. administer topically said pharmaceutical composition.

31. The use of claim 30, wherein step (a) is performed not more than one hour prior to step (b).

32. The use of claim 30, wherein pressure is applied on said first unit to allow said liquid to mix with said drug.

33. The use of claim 30, wherein said pressure is applied by the teeth and said pharmaceutical composition is administered on oral mucousa in the mouth cavity.

34. The use of claim 30 wherein said units are made from digestible materials, candy, guar-gum, bubble-gum, gelatin, or any combination thereof

35. The use of claim 30, wherein said liquid is water and isotonic saline.

36. The use of claim 30, wherein said liquid is an aqueous solution.

37. The use of claim 30, wherein said liquid is an aqueous solution comprising isotonic saline solution and water in a range of between 1:1 to 8:1 ratio.

38. The use of claim 30, wherein said liquid is an aqueous solution comprising a second drug.

39. The use of claim 30, wherein said drug is powder.

40. The use of claim 30, wherein said drug is sucralfate.

41. The use of claim 30, wherein said liquid is an aqueous solution comprising an isotonic saline solution and water in a range of between 1:1 to 8:1 ratio, and said drug is sucralfate.

42. The use of claim 30, for treating an ulcer of Aphthous Stomatitis, in a subject, comprising the steps of mouth rinse followed by topically administration of said pharmaceutical composition, wherein said drug is sucralfate, and said liquid is an aqueous solution.

43. The use of claim 30, for treating an ulcer of Aphthous Stomatitis in a subject suffering from Oral Mucositis, induced by chemotherapy and radiotherapy, comprising the steps of mouth rinse followed by topically administration of said pharmaceutical composition, wherein said drug is sucralfate, and said liquid is an aqueous solution.

44. The use of claim 30, for treating an ulcer of Oral Lichen Planus in a subject, comprising the steps of applying a rinsing fluid on said ulcer followed by topically administration said pharmaceutical composition, wherein said drug is sucralfate, and said liquid is an aqueous solution.

45. The use of claim 42, wherein the concentration of said sucralfate is in a range of ½-10 gr in about 2-5 mL water and isotonic saline solution in 1:1 ratio.

46. The use of claim 42, wherein said Aphthous Stomatitis is Recurrent Aphthous Stomatiti (R.A.S), or Oral Mucositis or combination thereof.

47. The use of claim 42, wherein said Aphthous Stomatitis occur as a minor Aphthae, major Aphthae or Herpetiform Lesions.

48. A topical composition for topical treatment for ulcers of Aphthous Stomatitis comprising sucralfate in an aqueous solution or paste, wherein the concentration of said sucralfate is in a range of ½-10 gr in about 2-5 mL water and isotonic saline in a 1:1 ratio and freshly prepared and freshly administered.

49. A composition according to claim 48, wherein said concentration of said sucralfate is about 2 gr sucralfate in a range of between about 1-2 mL water and isotonic saline in 1:1 ratio.

50. The composition according to claim 48, wherein said composition is in a form of a paste, cream, gel, ointment or lotion.

51. The composition according to claim 48, further comprising aromatic oil.

52. The composition of claim 48, wherein said aromatic oil is sweet marjoram, myrrh, vanilla, lavender, juniper, clove bud or any combinations thereof.

53. The composition according to claim 48, wherein said water are mineral water.

54. The composition according to claim 48, wherein said Aphthous Stomatitis is Recurrent Aphthous Stomatitis (R.A.S).

55. The composition according to claim 48, wherein said Aphthous Stomatitis occur as a minor Aphthae, major Aphthae or Herpetiform Lesions.

56. A kit for treating ulcers of Aphthous Stomatitis comprising sucralfate powder.

57. The kit according to claim 56, wherein said powder is kept is a sealed container

58. The kit according to claim 56, wherein said container is opaque.

59. The kit according to claim 56, wherein said container is divided into portions wherein each portion contains a predetermined amount of said powder.

60. The kit according to claim 56, further comprises an aqueous solution wherein said solution is separated from said powder.

61. The kit according to claim 60, wherein said aqueous solution is divided into portions wherein each portion contains a predetermined amount of said solution.

62. The kit according to claim 60, wherein said aqueous solution is mineral water or tap water and isotonic saline.

63. The kit according to claim 56, further comprising an applicator, and a rinsing fluid.

64. A method of treating ulcer of Aphthous Stomatitis, periodontal pockets, abscesses, site of extractions or any combination thereof in a subject, comprising the steps of:
a. mouth-rinse
b. mixing a powder comprising sucralfate with water to obtain a mixture; and
c. topically administering said fresh mixture to said ulcer and surrounding mucosa;
thereby treating an ulcer of Aphthous Stomatitis in said subject.

65. The method according to claim 64, wherein the concentration of said sucralfate is in a range of ½-10 gr in about 2-5 mL water and isotonic saline in a 1:1 ratio.

66. The method according to claim 64, wherein the concentration of said sucralfate is about 2 gr in a range of between about 1-2 mL water and saline solution in a 1:1 ratio.

67. The method according to claim 64, wherein step (b) is performed not more than one hour prior to step (c).

68. A method of preventing the recurrence of an ulcer of Aphthous Stomatitis in a subject suffering from an ulcer of Aphthous Stomatitis, comprising the steps of;
a. mouth rinse
b. Mixing a powder comprising sucralfate with water to obtain a mixture; and
c. topically administering said fresh mixture to said ulcer site in the prodrome and to the surrounding mucosa;
thereby preventing the recurrence of an ulcer of Aphthous Stomatitis in said subject.

69. The method according to claim 68, wherein the concentration of said sucralfate is in a range of ½-1 gr in about 2-5 mL water and isotonic saline in a 1:1 ratio.

70. The method according to claim 68 wherein the concentration of said sucralfate is about 2 gr in a range of between about 1-2 mL water and isotonic saline in a 1:1 ratio.

71. The method according to claim 68, wherein step (b) is performed not more than one hour prior to step (c).

72. A method of treatment of ulcer of Aphthous Stomatitis in a subject suffering from Oral Mucositis, induced by chemotherapy and radiotherapy, comprising the steps of;
a. mouth rinse
b. mixing a powder which includes sucralfate with water and isotonic saline to obtain a mixture; and
c. topically freshly administering said mixture to said ulcer and surrounding mucosa;
thereby treating an ulcer of Oral Mucositis, induced by chemotherapy and radiotherapy.

73. A method of treatment an ulcer of Oral Lichen Planus in a subject, comprising the steps of;
a. mouth rinse;
b. mixing a powder which includes sucralfate with water and isotonic saline to obtain a mixture; and
c. topically freshly administering said mixture to said ulcer and surrounding mucosa;
thereby treating an ulcer of Oral Lichen Planus in a subject.
